(19) United States
(12) Patent Application Publication
(10) Pub. No.: US 2015/0239225 A1
(43) Pub. Date: Aug. 27, 2015

(54) Method for Laser Welding a Medical Device

(71) Applicant: SANOFI-AVENTIS DEUTSCHLAND GMBH, Frankfurt am Main (DE)

(72) Inventors: Michael Helmer, Frankfurt am Main (DE); Christoph Eissengarthen, Ginsheim (DE); Winfried Huthmacher, Frankfurt (DE); Carsten Mosebach, Mainz (DE)

(73) Assignee: Sanofi-Aventis Deutschland GmbH, Frankfurt am Main (DE)

(21) Appl. No.: 14/430,058

(22) PCT Filed: Sep. 19, 2013
(86) PCT No.: PCT/EP2013/069445

§ 371 Date: Mar. 20, 2015

(30) Foreign Application Priority Data
Sep. 24, 2012 (EP) 12185577.9

(51) Int. Cl. B32B 38/00; B32B 37/06; A61M 5/178

(52) U.S. Cl. CPC B32B 38/0008; A61M 5/178; B32B 37/06; A61M 2207/00; B32B 2310/0843; B32B 2398/20; B32B 2535/00

(57) ABSTRACT

A method of welding two parts of a medical device is provided, comprising the steps of providing a first part having an internal surface with a provided welding seam and providing a second part having a joining portion comprising an external surface with an opposite welding seam, wherein at least one of the first and the second part has one or more tensioning members that are configured to interact with the surface of the other one of the first and second part to create a normal force between the welding seam of the first part and opposite welding seam of the second part, and comprising the step of inserting the joining portion of the second part inside of the first part locating at least on welding location and comprising the step of delivering at least one pulse of laser beam energy, each pulse being directed to the welding location.

### CROSS REFERENCE TO RELATED APPLICATIONS

The present application is a U.S. National Phase Application pursuant to 35 U.S.C. §371 of International Application No. PCT/EP2013/069445 filed Sep. 19, 2013, which claims priority to European Patent Application No. 12185577.9 filed Sep. 24, 2012. The entire disclosure contents of these applications are herewith incorporated by reference into the present application.

### FIELD OF INVENTION

The disclosure relates to structural plastic members integral to a medical device part that may create location insensitive joining forces when combined to a second part to allow laser welding of the two parts and to a method for laser welding medical device parts; in particular, methods and apparatus for welding disposable injection device components.

### BACKGROUND

Laser welding has gained widespread acceptance in the industry, producing welds for items ranging from cigarette lighters and watch springs, to medical devices and related components, such as pacemakers, implantable defibrillators, batteries and hybrid circuit packages.

Welding requires heating materials to a molten state so that they become fused together. A laser may be employed to generate light energy that can be concentrated and absorbed at a location in materials, producing the heat energy necessary to perform the welding operation. By using light energy in the visible or infrared portions of the electromagnetic spectrum, energy can be directed from its source to the material to be welded using optics that can focus and direct the energy with the required amount of precision. After the applied light energy is removed, the molten material solidifies and then begins to slowly cool to the temperature of the surrounding material.

The type of weld may have an influence on the laser welding parameters and the stability of the welding process. There are two general weld types—seam welds and spot-welds. Seam welding forms a continuous weld, while spot welding consists of discrete weld locations. Likewise, there can be direct laser welding and transmission laser welding.

Laser welding systems typically consist of a laser source, a beam delivery system, and a workstation. Carbon Dioxide (CO2) and Nd:YAG (Neodymium-doped Yttrium Aluminum Garnet) are two laser sources or laser media used for laser welding applications. Frequently, semiconductor lasers are used. Both YAG and CO2 lasers may be used for seam welding and spot welding of both butt joints and lap (overlap) joints. Solid state lasers (which includes Nd:YAG, Nd:Glass and similar lasers), are often employed in low- to medium-power applications, such as those needed to quasi-weld, simultaneous weld or beam lead weld integrated circuits to thin film interconnecting circuits on a substrate, and similar applications.

For precise or delicate welding operations, solid state welding systems may offer the advantage of coaxial viewing optics that provide magnification so that the exact spot of the laser beam focus can be easily seen. This may enable more precise alignment and focusing of the laser beam, as well as work piece viewing. Since the wavelength of the Nd:YAG laser is close to the visible spectrum, optical lenses may be used to transmit both the laser light and the image of the work piece.

In certain welding applications requiring relatively low heat input (due to proximity to thermally-sensitive components, for example), the pulsed laser mode of operation may be suitable. When laser energy is absorbed by the material being welded, heat is conducted into the material, creating a weld pool in a very localized area. Depending on the type of material, some heat may be conducted through the part being welded and away from the weld zone, potentially toward thermally-sensitive material.

Transmission laser welding is now widely used for joining thermoplastics in industry, using laser sources with wavelengths from 0.8-1.1μm, such as diode, Nd:YAG and fiber lasers. The radiation at these wavelengths is less readily absorbed by natural plastics. Laser absorbing additives are therefore put into the lower part or applied as a thin surface coating at the joint. The parts are positioned together and tensioned before welding. The laser beam passes through the upper part to heat the joint at the absorbing surface of the lower part. The absorber in or on the lower plastic is typically carbon or an infrared absorber with minimal visible color, which allows a wide range of part colors and appearances to be welded. Transmission laser welding is capable of welding thicker parts than direct welding, and since the heat affected zone is confined to the joint region no marking of the outer surfaces occurs.

The amount of time required to perform certain welding operations imposes a constraint on manufacturing such devices due to the need to maintain a relatively low heat input to the device, as well as the need to achieve the degree of weld overlap necessary to achieve a sufficient weld. Additionally, the physical size of device components and the geometric relationship of the parts can also affect welding efficiency and hence manufacturing time. Minimizing manufacturing time through efficient and timely welding methods increases device production rates and reduces manufacturing costs.

The problem to be solved by the present invention is to provide an improved method for welding two parts of a medical device

### SUMMARY

Certain embodiments provide structural plastic components, in particular, structural biasing components, of plastic device parts to create the required joining forces when the parts are joined together without having to use an external device during manufacture to create these forces. The structural plastic components may be provided integral to the parts being joined. After being joined together, the parts may be welded. A method of laser welding that employs a plurality of these structural biasing components on medical device parts to be welded may lead to faster cycle times and may increase manufacturing throughput. Further, such methods may not rely on external devices to create the required joining forces.

Certain embodiments include a method of laser welding that employs one or more localized biasing members, in particular tensioning members, on one of the device parts to impart the required tensioning between two parts to be welded together. The tensioning members may create normal forces between the two parts to be welded together.

Certain embodiments of the disclosure provide cooperating tensioning members on a first and a second part, in particular a cartridge holder and a body housing of a disposable injection device, to create the necessary tensioning needed to achieve a laser weld during a high speed assembly process.

According to one aspect, a method of welding two parts of a medical device is provided, comprising the steps of providing a first part having an internal surface with a provided welding seam and providing a second part having a joining portion comprising an external surface with an opposite welding seam, wherein at least one of the first and the second part has one or more tensioning members that are configured to interact with the surface of the other one of the first and second part to create a normal force between the welding seam of the first part and opposite welding seam of the second part, and comprising the step of inserting the joining portion of the second part inside of the first part, and comprising the step of locating at least one welding location. Furthermore, the method comprises the step of delivering at least one pulse of laser beam energy, each pulse being directed to the welding location.

For example, the first and the second part may be parts of a housing of a drug delivery device. In particular, the first part may be a cartridge holder, and the second part may be a body housing. The first and the second part may be configured to be parts of a disposable injection medical device. At least one of the first part and the second part may be cylindrical.

The one or more tensioning members may create a normal force between the first part and the second part. The one or more tensioning members may be an integral part of one of the first and the second part. In particular, the one or more tensioning members may be positioned at the internal surface of the first part or at the external surface of the second part or at both. For example, one tensioning member may be located at the internal surface of the first part and one tensioning member may be located at the external surface of the second part. Alternatively, two tensioning members may be located at the external surface of the second part and none may be located at the internal surface of the first part. Alternatively, any other number and distribution of the one or more tensioning members may be possible.

According to one embodiment of the method, the at least one tensioning member may be located at a different location than the welding seams. Alternatively, the at least one tensioning member may be positioned at the same location as one of the welding seams.

The at least one tensioning member may comprise one or more protrusions. The protrusion may be configured as a raised rib. The rib may extend from a distal position towards a proximal position.

According to one embodiment of the method, the tensioning member, in particular the protrusion may comprise an insertion point to facilitate the assembling of the first and the second part. In particular, the insertion point may comprise a chamfered surface at one end of the tensioning member. The insertion point may be configured to facilitate the insertion of the second part in the first part.

According to one embodiment of the method, one or both of the first and the second part may be deformed by a force created by the at least one tensioning member. For example, the interaction of at least one tensioning member with the surface of one of the first or the second part may lead to a deformation of the part which does not comprise a tensioning member, in particular at the location of the welding seam. Additionally or alternatively, a deformation of the part comprising the tensioning member may occur. Due to the deformation, normal forces between the welding seam and the opposite welding seam are created.

The first and the second part may further comprise insertion members to ensure a proper orientation of the parts. The insertion members may engage in channels on the surface of the other one of the first and the second part.

Preferably, the parts being welded are not subject to a mating force generated by an external device.

These and other descriptions and depicted embodiments for fabricating welded medical devices are described in the following and the appended drawings and claims.

The term “medicament”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H—His—Gly—Glu—Gly—Thr—Phe—Thr—Ser—Asp—Leu—Ser—Lys—Gln—Met—Glu—Glu—Glu—Ala—Val—Arg—Leu—Phe—Ile—Glu—Trp—Leu—Lys—Asn—Gly—Gly—Pro—Ser—Ser—Gly—Ala—Pro—Pro—Pro—Ser—NH2.

Exendin-4 derivatives are for example selected from the following list of compounds:

H—(Lys)4-des Pro36, des Pro37 Exendin-4(1-39)-NH2,

H—(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38[Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [ Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38[Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof An example of a pharmaceutically acceptable salt of a poly-sulphated low molecular weight heparin is enoxaparin sodium.

Antibodies are globular plasma proteins (˜150 kDahttp://en.wikipedia.org/wiki/Dalton_{—}%28unit%29) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab')2 fragment containing both Fab pieces and the hinge region, including the H-H interchain disulfide bond. F(ab')2 is divalent for antigen binding. The disulfide bond of F(ab')2 may be cleaved in order to obtain Fab'. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### BRIEF DESCRIPTION OF THE DRAWINGS

FIG. 1 is a simplified schematic view of a disposable injection medical device completely assembled and unassembled in a sectioned view.

FIG. 2 is a graphic illustration of a transmission welding process.

FIG. 3 is a perspective view of one embodiment illustrating tension members extending distally from a second part and showing the second part in the process of being assembled to the first part by inserting the joining portion of the second part inside the first part. The following step will be welding together the first and second part.

### DETAILED DESCRIPTION

The following description of certain illustrated embodiments of the invention is presented to enable a person skilled in the art to appreciate certain aspects of the invention, including insubstantial modifications thereof, and to make and use the invention, as depicted and described as well as to illustrate other aspects of the invention. Various modifications to the illustrated embodiments will be readily apparent to those skilled in the art, and the generic principles herein may be applied to other embodiments and applications without departing from the spirit and scope of the invention as defined by the appended claims. Thus, the present invention is not intended to be limited to the embodiments shown, but is to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the invention. Skilled persons will recognize the examples provided herein have many useful alternatives that fall within the scope of the invention.

For purposes of illustration only, the embodiments of the invention are described below in the context of a disposable injection device, specifically the welding of a plastic cartridge holder to a plastic body housing that contains dose setting and dose delivery mechanisms. However, embodiments of the invention are not limited to injection devices, and may be employed in many various types of electronic and mechanical devices for treating patient medical conditions such as portable pumps, pacemakers, neurostimulators, and other therapeutic substance delivery systems.

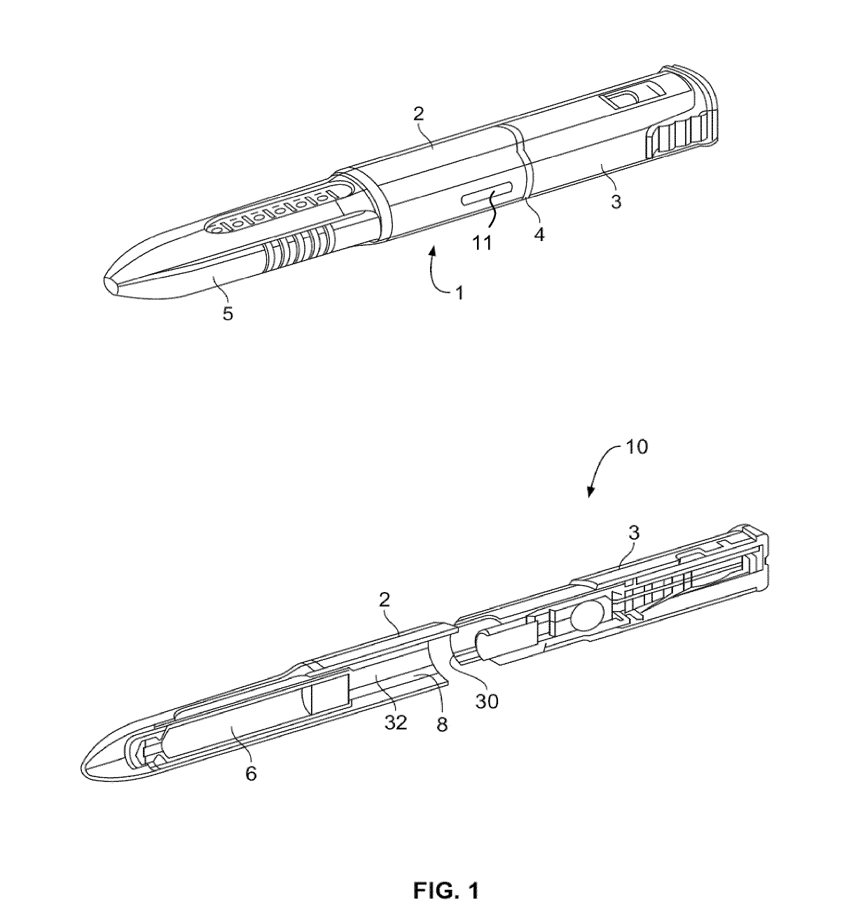

FIG. 1 is a simplified schematic view of an example of a disposable injection device, in particular an injection pen, in a fully assembled state 1 and in a sectional view in an un-assembled state 10. The pen has a first part 2 being a cartridge holder section, which contains a cartridge of medicament 6. The injection device 1 also has a second part 3 being a body housing section that encloses the dose setting and dose delivery mechanism. The first part 2 and the second part 3 are connected together during manufacture and assembly and share a common seam 4. The seam 4 may serve as a stop surface during an assembly of the first and the second part 2, 3. The first part 2 and the second part 3 are configured to be connected together by laser welding. For example, a transmission laser may be used. The laser may be directed to a welding location 11. Preferably, the first and the second part 2, 3 are welded in at least in an area of a welding seam 32 and an opposite welding seam 35.

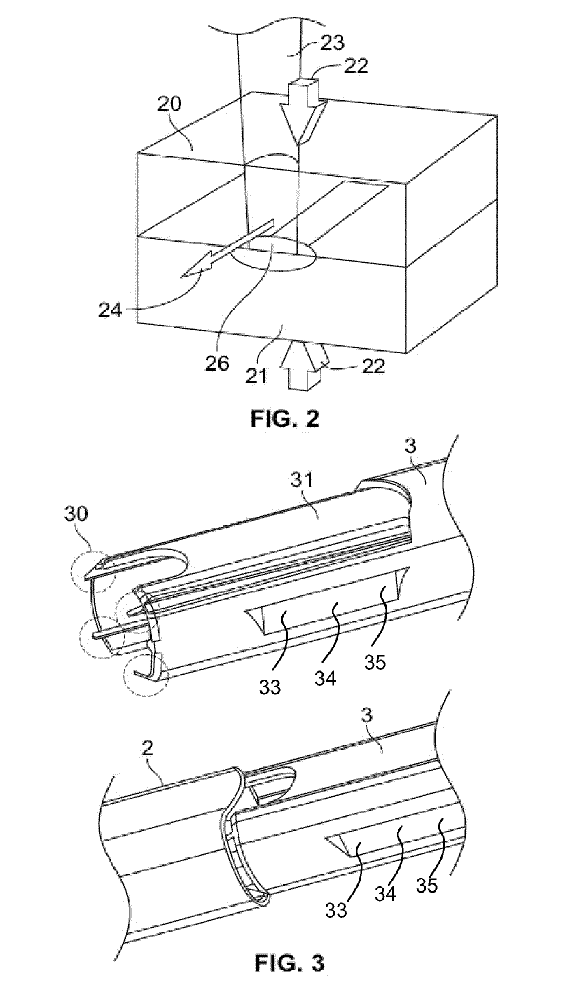

FIG. 2 is an illustration of how a transmission laser is used to weld two plastic parts 20 and 21, where the top part 20 is transparent to laser beam 23 and the bottom part 21 can be either transparent or opaque to the laser beam 23. The parts 20 and 21 may be first and second parts 2, 3. For example, the bottom part 21 comprises a material which absorbs the laser beam 23. The laser-absorbing material is located in an area where welding is intended. The main material of the bottom part 21 may be laser-absorbing. For example, the bottom part 21 comprises infra-red-absorbing plastic material. Alternatively or additionally, the bottom part 21 can be provided with a laser-absorbing material, for example during manufacturing of the bottom part 21, in particular by two-component injection molding. The laser absorbing material may be provided at least in a defined area of the bottom part 21. A weld zone 26 is created at the point where an absorbing material, for example an infra-red absorber, is located on the bottom part 21.

Arrow 24 indicates the path of the laser beam 23 in those situations where a continuous weld is desired. Alternatively, contour welding, quasi-simultaneous welding or simultaneous laser welding can be performed. In order to obtain an acceptable weld the two parts 20 and 21 are clamped under pressure 22. Such an application pressure may be performed using an external device, such as a holding down mask or a pressure pad. The need to apply this pressure or normal force to the two parts using an external device slows down the manufacturing and assembly process. The present disclosure eliminates the need for this external force generating device to simplify the mass production process.

FIG. 3 illustrates sections of the first and the second part 2, 3. In this embodiment, the need for an external pressure device is eliminated by adding a tensioning member 33 to one or more parts such that when the parts are joined together the tensioning member 33 imparts a force normal to a welding location 11 to which the laser will be directed. In FIG. 3, the tensioning member 33 is located at the external surface of the second part 3, in particular at the same location as the welding seam 35. In principle, the tensioning members 33 can be located on either of the parts 2, 3 or on both. By changing the geometric sizes of one or both of the parts the required mating forces may be provided, but in certain medical devices, such as injection devices, the geometric distortions can lead to improper functioning of the device. The tensioning members 33 of the present embodiments are preferably one or more protrusions 34, in particular ribs, that generate location-insensitive joining forces. “Location-insensitive” may mean that there is no need of a fine adjustment of the location of these protrusions 34. Instead, the required tensioning forces may primarily by adjusted by the height of the protrusions 34. In the described embodiment two almost concentric parts, in particular the first and the second part 2, 3 are assembled by inserting one into the other. A small gap is located between the parts in the joining area. The tensioning members 33, in particular the protrusions 34 are protruding from the external surface of the second part 3. Their height is larger than the size of the gap.

An interaction of the at least one tensioning member 33 with the internal surface of the first part 2 leads to a deformation of the first part 2 and/or the second part 3. The parts 2, 3 may be deformed out of their semi-concentric shape, particularly by the joining forces generated by the tensioning members 33, in particular the protrusions 34. This deformation could also occur with box shaped parts and flat walls. The force of an elastic strain of the components provides the joining force.

The first and the second part 2, 3 are deformed such that the gap between the first and second part 2, 3 is closed at least at a location where welding may occur. In one embodiment, the first and the second part 2, 3 may be deformed such that they are in mechanical contact at surfaces where no tensioning member 33, in particular no protrusion 34 is located. As an example, the second part 3 may be deformed inwardly at the location where a tensioning member 33 is located due to the tensioning force which is exerted on the second part 3 by the tensioning member 33. Furthermore, at the location where no tensioning member 33 is present, the second part 3 may be deformed outwardly to make up the inward deformation. In particular, the second part 3 may be deformed outwardly in a direction which is perpendicular to the tensioning force created by the tensioning members 33. Alternatively or additionally, the first part 2 may be deformed outwardly at the location where a tensioning member 33 is present due to the force which is exerted on the first part 2 by the tensioning member. To make up the outward deformation, the first part 2 may be deformed inwardly at a location where no tensioning member 33 is present. In particular, the first part 2 may be deformed inwardly in a direction which is perpendicular to the tensioning force created by the tensioning members 33. Due to the deformation of one or both parts 2, 3, the parts 2, 3 are pressed together. In particular, a normal force is created between the welding seams at a location where no tensioning member 33 is present. Thereby, a welding at the compressed location is possible.

The second part 3 may have a number of tensioning members 33, in particular protrusions 34 spaced radially around the outer circumference of the joining section 31 of the second part 3. The tensioning members 33, in particular the protrusions 34, may be positioned at a different location than a welding seam 32, 35. Alternatively, the tensioning members 33, in particular the protrusions 34, may be positioned at the same location than the welding seam 32, 35. These protrusion 34 preferably are slightly raised such that they exert a normal tensioning force on the inside circumference 32 (see FIG. 1) of the first part 2. The length and thickness of the protrusions 34 determine the possible longitudinal locations where welding can occur.

To assist in joining the first and the second part 2, 3, it may be desired to use insertion members 30. This is especially helpful where one part has undergone a shape distortion. Likewise, a plurality of channels or grooves 8 equaling the number of additional orientation or insertion members 30 may be included on the inside circumference 32 of the first part 2 to assist in the assembly process and to ensure proper orientation of the parts 2, 3. Preferably, the tensioning members 33 of the present disclosure are integral to at least one of the parts 2, 3 being joined and are preferably formed during the molding process used to make each part.

The protrusions 34 comprise insertion points that are configured to facilitate the assembling of the first and the second part 2, 3. In particular, an insertion point may be an end of a protrusion 34 which comprises a chamfered surface. For example, the second part 3 is configured to be inserted into the first part 2 with the chamfered surface of the protrusion ahead. Thereby, the second part 3 may easily slide into the first part 2 without fine adjustment being required. In an alternative embodiment, the insertion point may be a part of the first part 2, or both of the first and the second part 2, 3 may comprise insertion points.

Once the parts 2, 3 are joined and held in place, successive laser pulses can be delivered to given locations to create the weld. In certain embodiments of the present invention control of the delivery of laser energy to a particular location on a work-piece is possible through the use of a steered or directed laser beam system. Steered or directed laser beam systems move the laser beam with respect to the work-piece to direct laser energy to a particular location. These techniques have been developed for applications in which work-pieces need to be processed at relatively high speeds to be economical. In some cases the use of mirrors for reflecting laser beam energy to direct the focal point of the laser beam to a desired location on the work-piece is required. The position and angle of the mirrors is adjusted in the welding process, typically using a computer controlled system, to change the position of the focal point of the laser beam on the work-piece.

Laser energy may either be delivered to the work-piece and directed to a particular location by using optics such as focusing lenses, mirrors, etc., or a fiber-optic beam delivery (FOBD) system, or some combination thereof. When optics such as focusing lenses, mirrors, etc. are used, the laser may, for example, be positioned near the top of the workstation or fixture, and a mirror may be positioned at a certain angle and location relative to the laser source to direct the laser beam through a focusing lens and to a particular location on the work-piece. An FOBD system uses an optical cable to deliver the laser energy to the workstation, enabling the laser source to be located remotely from the work-piece during welding, if desired. FOBD systems may be configured to permit the output of one laser source to supply the laser energy to be used at several workstations in different locations.

Thus, select embodiments of the invention are herein disclosed, depicted and described. One skilled in the art will appreciate that the present invention can be practiced with embodiments other than those disclosed. The disclosed embodiments are presented for purposes of illustration and not limitation, and the present invention is limited only by the claims that follow, including insubstantial changes therefrom.

1-15. (canceled)

16. A method of welding two parts of a medical device, comprising,
a. providing a first part having an internal surface with a welding seam;
b. providing a second part having a joining portion comprising an external surface with an opposite welding seam, wherein at least one of the first and the second part has one or more tensioning members that are configured to interact with the surface of the other one of the first and second part to create a normal force between the welding seam of the first part and the opposite welding seam of the second part;
c. inserting the joining portion of the second part inside the first part;
d. locating at least one welding location; and
e. delivering at least one pulse of laser beam energy directed to the welding location; wherein the one or more tensioning members are positioned at a different location than the welding seams.

17. The laser welding method of claim 16, wherein the one or more tensioning members are integral parts of one of the first and the second part or of both.

18. The laser welding method of claim 16, wherein the one or more tensioning members are positioned at the internal surface of the first part or at the external surface of the second part or at both.

19. The laser welding method of claim 16, wherein the one or more tensioning members comprise one or more protrusions.

20. The laser welding method of claim 19, wherein the one or more protrusions are configured as raised ribs.

21. The laser welding method of claim 19, wherein the one or more protrusions comprise insertion points to facilitate the assembling of the first and the second part.

22. The laser welding method of claim 16, wherein one or both of the first and the second part are deformed by a joining force created by the one or more tensioning members.

23. The laser welding method of claim 22, wherein an interaction of the one or more tensioning members with the surface of one of the first or the second part leads to a deformation of at least the part which does not comprise a tensioning member 33, at least at the location of the welding seam.

24. The laser welding method of claim 22, wherein at least one of the first and the second part are deformed such that they are in mechanical contact at surfaces where no tensioning member is located.

25. The laser welding method of claim 22, wherein due to the deformation of one or both parts, the parts are pressed together and a normal force is created between the welding seams at a location where no tensioning member is present.

26. The laser welding method of claim 16, wherein one or both of the first and the second part comprise insertion members to ensure proper orientation of the parts.

27. The laser welding method of claim 16 wherein the first and the second part are configured to be parts of a disposable injection medical device.

28. The laser welding method of claim 16 wherein the parts being welded are not subject to a mating force generated by an external device.

29. The laser welding method of claim 16, wherein at least one of the first part and the second part is cylindrical.

30. The laser welding method of claim 16 wherein a gap is located between the first and the second part in the joining area, wherein the first and the second part are deformed such that the gap is closed at least at a location where welding may occur.
